(19) United States
(12) Patent Application Publication
(10) Pub. No.: US 2017/0367655 A1
(43) Pub. Date: Dec. 28, 2017

(54) STATE DETERMINATION SYSTEM

(71) Applicant: TS TECH CO., LTD., Saitama (JP)

(72) Inventor: Shinji SUGIYAMA, Tochigi (JP)

(21) Appl. No.: 15/539,311

(22) PCT Filed: Dec. 24, 2015
(86) PCT No.: PCT/JP2015/085957

§ 371 Date: Jun. 23, 2017

(30) Foreign Application Priority Data
Dec. 25, 2014 (JP) 2014-262819

(51) Int. Cl. A61B 5/00; A61B 5/08; A61B 5/18

(52) U.S. Cl. CPC A61B 5/6893; A61B 5/18; A61B 5/0816; A61B 2562/04

(57) ABSTRACT

Upon determining a physiological state of a seated person on the basis of measurement results of some of plural sensors, the sensors are easily and properly selected. A system includes sensors that measure measurement values to be changed on a seating surface of a seat, and a determination device. The determination device includes a storage unit that stores association relationships set by associating each area with some of the sensors, an area identification unit that identifies an area in which the coordinates of a gravity center of the seated person are located among the areas, a sensor selection unit that selects the sensors associated with the area identified by the area identification unit from the association relationships stored by the storage unit, and a determination unit that determines a physiological state of the seated person on the basis of the measurement results of the selected sensors are provided.

### TECHNICAL FIELD

The present invention relates to a state determination system that determines a physiological state of a seated person on a seat, and in particular, relates to a state determination system including plural sensors that measure measurement values to be changed in accordance with a physiological activity of the seated person.

### BACKGROUND ART

A state determination system that measures measurement values to be changed in accordance with a physiological activity of a seated person on a seating surface of a seat and determines a physiological state of the seated person on the basis of a measurement result thereof is already known. As one example of the state determination system, a system that detects breathing of a seated person (specifically, vibration of a body surface following the breathing) by a pressure sensor of measuring pressure applied to a seating surface of a vehicle seat and determines an awakening state of the seated person on the basis of a detection result thereof is known.

In some state determination systems, the sensors that measure measurement values described above are provided at plural points. In such a configuration that the plural sensors are provided, for example, measurement results of the respective sensors are aggregated, a representing value such as an average value is obtained, and the state is determined on the basis of the representing value. However, since the measurement results of all the plural sensors are aggregated, a processing load is accordingly increased. In addition, since arrangement positions of the plural sensors are different from each other, the measurement results of the sensors are varied in accordance with a position where the seated person actually sits on the seating surface or a posture of the seated person.

From the above situations, it is thought that in the state determination system including the plural sensors, a proper sensor is selected among the plural sensors and the state is determined on the basis of an aggregate result of only the selected sensor. A specific example will be described. In a state determination system described in Patent Literature 1 (called as the “awakening degree estimation device” in Patent Literature 1), a sensor determined to be in stable contact with a seated person is identified among plural sensors, and a state is determined on the basis of a measurement result of only the identified sensor. As another example, in a state determination system described in Patent Literature 2 (called as the “biological information acquisition system” in Patent Literature 2), evaluation values indicating whether or not heart beats of a seated person are precisely detected are calculated respectively for plural sensors, and a state is determined on the basis of a measurement result of only the sensor having the highest evaluation value.

### CITATION LIST

### Patent Literature

PATENT LITERATURE 1: JP 2013-172899 A

PATENT LITERATURE 2: JP 2013-99528 A

### SUMMARY OF INVENTION

### Technical Problem

In the state determination system including the plural sensors, in a case where some sensors are selected among the plural sensors and the state is determined on the basis of the measurement results of the sensors, the sensors are desirably easily and properly selected. However, in Patent Literature 1, a specific method of identifying the sensors to be selected (strictly speaking, the sensors determined to be in stable contact with the seated person) is not described. In Patent Literature 2, since the evaluation values are calculated respectively for the plural sensors upon selecting the sensor, a processing load of arithmetic processing or the like is increased.

Meanwhile, as described above, the measurement results when each of the plural sensors measures the measurement value at an arrangement point thereof is varied in accordance with the position where the seated person actually sits on the seating surface (hereinafter, referred to as the seated position) or the posture of the seated person. In addition, there is sometimes a case where the seated person moves his/her body or the like and the above seated position or the posture itself is changed. When some sensors are selected among the plural sensors in such a case, there is a need for selecting sensors once more in consideration with a seated position or a posture after the change.

The present invention is achieved in consideration with the above problems and an object thereof is to provide a system capable of easily and properly selecting sensors as a state determination system that selects some sensors among plural sensors and determines a physiological state of a seated person on the basis of measurement results of the sensors.

Another object of the present invention is, in a case where a seated person moves his/her body or the like and a seating state is changed, to select sensors in consideration with a seating state after the change.

### Solution to Problem

With a state determination system of the present invention, the above problems are solved by having sensors that measure measurement values to be changed in accordance with a physiological activity of a seated person on a seating surface of a seat, and a determination device that determines a physiological state of the seated person on the basis of measurement results of the sensors, wherein the plural sensors are arranged in such a manner that coordinates of arrangement positions of the sensors fixed when both the width direction and the front to back direction of the seat serve as the axial directions of the coordinate axes are different from each other, and the determination device has a storage unit that stores association relationships set by associating each of areas when the seating surface is partitioned into the plural areas with some of the plural sensors, an area identification unit that identifies in which area coordinates of a gravity center position of the seated person fixed when both the directions serve as the axial directions of the coordinate axes are placed among the plural areas, a sensor selection unit that selects the sensors associated with the area identified by the area identification unit among the plural sensors from the association relationships stored by the storage unit, and a determination unit that determines the physiological state on the basis of the measurement results of the sensors selected by the sensor selection unit.

In the state determination system of the present invention formed as above, the seating surface is partitioned into the plural areas, and each of the plural areas is associated with some of the plural sensors. Then, in which area the coordinates of the gravity center position of the seated person are placed among the plural areas is identified, and the sensors associated with the identified area are selected. The physiological state of the seated person is determined on the basis of the measurement results of only the selected sensors. In such a way, in the state determination system of the present invention, the gravity center position is used as an indicator showing a seating state of the seated person, and the sensors are selected on the basis of this gravity center position. Thus, the sensors thought to be proper in consideration with the seating state of the seated person can be selected. When the sensors are selected, the area to which the gravity center position belongs is identified and then the association relationship between the area and the sensors is read out from the storage unit and the association relationship is referenced, so that the sensors associated with the area to which the gravity center position belongs are selected. Thereby, proper sensors can be readily selected among the plural sensors.

In the above state determination system, favorably, the storage unit stores the association relationships set by associating each of the areas when the seating surface is partitioned in such a manner that two or more areas exist in each of both the directions with some of the plural sensors.

With the above configuration, the seating surface is partitioned in such a manner that two or more areas exist in each of the front to back direction and the width direction of the seat, and each area is associated with some of the plural sensors. Thereby, since the seating surface is more finely partitioned, the area to which the gravity center position belongs can be finely identified. As a result, even when the sensors associated with the area to which the gravity center position belongs are selected, the sensors can be more properly selected.

In the above state determination system, more favorably, the storage unit stores the association relationships set by associating each of the areas when the seating surface is partitioned in such a manner that the number of the areas positioned on the back side in the front to back direction is more than the number of the areas positioned on the front side with some of the plural sensors.

In the above configuration, the seating surface is partitioned in such a manner that the number of the areas positioned on the back side in the front to back direction is more than the number of the areas positioned on the front side. This reflects that the gravity center of the seated person is frequently positioned on the back side of the seating surface in general. When the number of the areas on the back side is more in such a way, the area to which the gravity center position belongs can be more finely identified. As a result, when the sensors associated with the area to which the gravity center position belongs are selected, the sensors can be more properly selected.

In the above state determination system, still more favorably, at least three or more of the sensors are arranged, and the storage unit stores the association relationships set by associating each of the areas with two of the sensors.

In the above configuration, each area is associated with the two sensors. That is, the measurement results of the sensors adopted at the time of state determination become the measurement results of the two sensors. With such a configuration, even if an abnormality is caused in one of the two sensors but the other sensor is normal, the physiological state of the seated person can be properly determined on the basis of the measurement result of the sensor.

In the above state determination system, further favorably, the storage unit stores the association relationships set by associating each of the areas with two of the sensors among the plural sensors, the two sensors whose coordinates of the arrangement positions straddle the coordinates of the gravity center position in the width direction when the coordinates of the gravity center position are placed in each of the areas.

In the above configuration, when the coordinates of the gravity center position are placed in each area, the two sensors arranged at the positions straddling the coordinates of the gravity center position are selected. In general, there is a tendency that the measurement results of the two sensors among the plural sensors arranged at the positions straddling the gravity center position have a correlation with each other. Therefore, when the respective measurement results of the two sensors arranged at the positions straddling the coordinates of the gravity center position are used, the physiological state of the seated person can be more properly determined.

In the above state determination system, even more favorably, the storage unit stores the association relationships set by associating each of the areas when the seating surface is partitioned in such a manner that the areas exist symmetrically with respect to a center position of the seating surface in the width direction with some of the plural sensors.

In the above configuration, the seating surface is partitioned in such a manner that the areas exist symmetrically with respect to the center position of the seating surface in the width direction of the seat, and each area is associated with some of the plural sensors. With such a configuration, symmetry of area arrangement is utilized, so that identification of the area to which the gravity center position belongs and selection of the sensors associated with the area can be more readily performed.

In the above state determination system, more and more favorably, the storage unit stores the association relationships set by associating each of the areas when the seating surface is partitioned in such a manner that length in the width direction of the area closer to a center position of the seating surface among at least three or more of the areas placed side by side in the width direction is shorter than the length of the area more distant from the center position with some of the plural sensors.

In the above configuration, the seating surface is partitioned in such a manner that the area closer to the center position of the seating surface among at least three or more of the areas placed side by side in the width direction of the seat is narrower than the area more distant from the center position, and each area is associated with some of the plural sensors. Since the area closer to the center position of the seating surface among the areas placed side by side in the width direction is narrower in such a way, the area to which the gravity center position belongs can be more finely identified in the vicinity of the center position. As a result, when the sensors associated with the area to which the gravity center position belongs are selected, more proper sensors can be selected.

In the above state determination system, furthermore favorably, the number of the sensors arranged on the back side in the front to back direction is more than the number of the sensors arranged on the front side.

In the above configuration, the number of the sensors arranged on the back side in the front to back direction of the seat is more than the number of the sensors arranged on the front side. This reflects that the gravity center of the seated person is frequently positioned on the back side of the seating surface in general. When the arrangement number of the sensors is more on the back side in such a way, as the sensors associated with the area to which the gravity center position belongs, more proper sensors can be selected.

In the above state determination system, further favorably, four of the sensors are arranged symmetrically with respect to the center position of the seating surface in the width direction on the back side in the front to back direction, and two of the sensors are arranged symmetrically with respect to the center position in the width direction on the front side.

In the above configuration, the four sensors are arranged on the back side in the front to back direction of the seat and the two sensors are arranged on the front side, respectively symmetrically with respect to the center position in the width direction of the seating surface. Since the arrangement number of the sensors is more on the back side of the seating surface and the sensors are arranged symmetrically with respect to the center position in the width direction of the seating surface in such a way, as the sensors associated with the area to which the gravity center position belongs, more proper sensors can be selected.

In the above state determination system, favorably, the storage unit stores the association relationships set by associating each of the areas with a sensor among the plural sensors, the sensor of a higher reflection degree on the measurement result of the measurement value of the physiological activity when the coordinates of the gravity center position are placed in each of the areas.

In the above configuration, each of the areas is associated with the sensor of the higher reflection degree on the measurement result of the measurement value of the physiological activity when the coordinates of the gravity center position are placed in each of the areas. Thereby, when the sensor associated with the area to which the gravity center position belongs is selected on the basis of the above association relationship, a proper sensor can be selected.

In the above state determination system, favorably, the determination device further has a change degree identification unit that identifies a change degree of the measurement results of the sensors selected by the sensor selection unit, when a magnitude of the change degree identified by the change degree identification unit exceeds a threshold value, the area identification unit re-identifies the area, and accordingly the sensor selection unit re-selects some of the sensors, and then the determination unit determines the physiological state on the basis of measurement results of only the re-selected sensors, and when the magnitude of the change degree identified by the change degree identification unit falls below the threshold value, the determination unit determines the physiological state on the basis of the measurement results of only the sensors selected by the sensor selection unit before the change degree identification unit identifies the change degree.

In the above configuration, in a case where the measurement results of the sensors selected by the sensor selection unit are remarkably changed, sensors are selected and the state is determined on the basis of measurement results of the re-selected sensors. With such a configuration, even when the seated person moves and the gravity center position is changed, by re-selecting the sensors, the sensors associated with the area to which the gravity center position after the change belongs can be selected.

Meanwhile, in a case where no remarkable change is seen in the measurement results of the sensors selected by the sensor selection unit, the measurement results of the sensors which have been used until then are continuously utilized, and the state is determined on the basis of the measurement results. That is, while the gravity center position is not changed, the measurement results of the same sensors continue to be used. Thus, time and labor of re-selecting the sensors can be omitted.

### Advantageous Effects of Invention

According to the present invention, upon determining the physiological state of the seated person on the basis of the measurement results of some of the plural sensors, the sensors can be easily and properly selected.

In addition, according to the present invention, by partitioning the seating surface in such a manner that two or more areas exist in each of the front to back direction and the width direction of the seat, the area to which the gravity center position belongs can be finely identified. As a result, the sensors can be more properly selected.

In addition, according to the present invention, by partitioning the seating surface in such a manner that the number of the areas positioned on the back side is more than the number of the areas positioned on the front side, the area to which the gravity center position belongs is more finely identified, so that more proper sensors can be selected.

In addition, according to the present invention, by associating each area with the two sensors, even if an abnormality is caused in one of the two sensors, the physiological state of the seated person can be properly determined.

In addition, according to the present invention, by selecting the two sensors arranged at the positions straddling the coordinates of the gravity center position when the coordinates of the gravity center position are placed in each area, the physiological state of the seated person can be more properly determined.

In addition, according to the present invention, by partitioning the seating surface in such a manner that the areas exist symmetrically with respect to the center position of the seating surface in the width direction of the seat, the symmetry of the area arrangement is utilized, so that identification of the area to which the gravity center position belongs and selection of the sensors can be more readily performed.

In addition, according to the present invention, since the area closer to the center position of the seating surface among the areas placed side by side in the width direction is narrower, the area to which the gravity center position belongs is more finely identified in the vicinity of the center position, so that more proper sensors can be selected.

In addition, according to the present invention, since the number of the sensors arranged on the back side is more than the number of the sensors arranged on the front side, more proper sensors can be selected.

In addition, according to the present invention, since the four sensors are arranged on the back side and the two sensors are arranged on the front side, respectively symmetrically with respect to the center position in the width direction of the seating surface, more proper sensors can be selected.

In addition, according to the present invention, since each of the areas is associated with the sensor of the higher reflection degree on the measurement result of the measurement value of the physiological activity when the coordinates of the gravity center position are placed in each of the areas, a proper sensor can be selected.

In addition, according to the present invention, even when the seated person moves and the gravity center position is changed, by re-selecting the sensors, the sensors associated with the area to which the gravity center position after the change belongs can be selected. Meanwhile, by continuously using the measurement results of the same sensors while the gravity center position is not changed, time and labor of re-selecting the sensors can be omitted.

### BRIEF DESCRIPTION OF DRAWINGS

FIG. 1 is a diagram showing the entire configuration of a state determination system according to one embodiment of the present invention.

FIG. 2 is a diagram showing a configuration of a determination device from a functional aspect.

FIG. 3 is a plan diagram showing arrangement of sensors.

FIG. 4 is a graph showing measurement results of the sensors.

FIG. 5 is a diagram showing positions of areas when a seating surface is partitioned into the plural areas.

FIG. 6 is a diagram showing relationships between a displacement amount of a chest portion following breathing and the measurement results of the sensors.

FIG. 7 is a diagram showing an experiment result relating to a reflection sensor when coordinates of a gravity center position are changed.

FIG. 8 is a diagram showing association relationships between the areas and the reflection sensor.

FIG. 9 is a diagram showing the measurement result of the sensor and a change degree thereof.

FIG. 10 is a diagram showing a flow of a state determination flow (No. 1)

FIG. 11 is a diagram showing the flow of the state determination flow (No. 2)

FIG. 12 is a diagram showing association relationships between areas and sensors in a modified example.

FIG. 13 is a diagram showing a flow of a state determination flow.

### DESCRIPTION OF EMBODIMENTS

<<State Determination System According to One Embodiment of the Present Invention>>

Hereinafter, a state determination system according to one embodiment of the present invention (the present embodiment) will be described. A vehicle seat mounted on a vehicle will be described as one example of a seat below. In the following description, the “front to back direction” corresponds to the front to back direction of the seat, indicating the front to back direction when seen from a seated person sitting on the vehicle seat, specifically the front to back direction of the vehicle (in other words, the travelling direction). The “width direction” corresponds to the width direction of the seat, indicating the right and left direction when seen from the seated person sitting on the vehicle seat.

The state determination system according to the present embodiment (hereinafter, referred to as the present system 10) is a system for determining (estimating) a physiological state of the seated person of the vehicle seat. In the present embodiment, the “physiological state” indicates an awakening state of the seated person but is not limited to this. The “physiological state” indicates not only the awakening state but also a state relating to a normality/abnormality of works and functions of body portions (including internal organs and nerves) of the seated person such as a mood stabilized state and a heavily intoxicated state of the seated person. The present invention can be applied to a system that determines such a state.

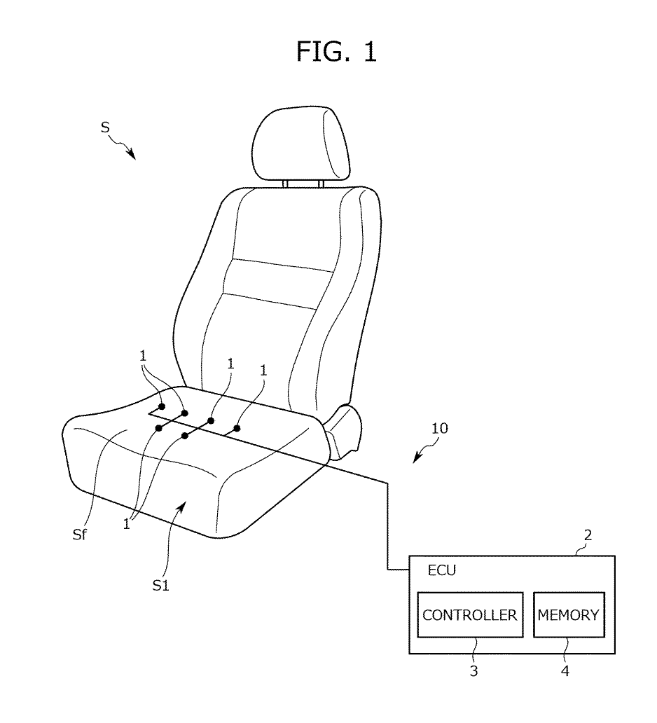

A configuration of the present system 10 will be described with reference to FIG. 1. The present system 10 is formed by breathing sensors 1 serving as sensors, and an ECU (Electronic Control Unit) 2 serving as a determination device. FIG. 1 is a diagram conceptually showing the entire configuration of the present system 10.

The breathing sensors 1 are known pressure sensors such as piezo-sensor type pressure sensors, semiconductor piezoresistance type pressure sensors, strain gauge type pressure sensors, electrostatic capacitance type pressure sensors, or silicon resonant type pressure sensors. The breathing sensors 1 are provided in a seat cushion S1 of a vehicle seat S, more specifically in a seating portion of the seat cushion S1 (portion supporting the buttocks of the seated person).

The breathing sensors 1 measure pressure (seating pressure) applied to an upper surface of the seat cushion S1, that is, a seating surface Sf when the seated person sits on the vehicle seat S. The seating pressure is periodically changed in accordance with a physiological activity, specifically breathing of the seated person sitting on the vehicle seat S. That is, the breathing sensors 1 measure the seating pressure to be changed in accordance with breathing of the seated person on the seating surface Sf as measurement values. The breathing sensors 1 are arranged at positions immediately below the seating surface Sf upon measuring the seating pressure, strictly speaking, arranged at positions sandwiched between a cushion pad forming the seat cushion S1 and a skin material covering the cushion pad. However, the present invention is not limited to this but the breathing sensors 1 may be arranged at positions where the seating pressure can be properly measured such as on the seating surface Sf of the seat cushion S1.

In the present embodiment, the plural breathing sensors 1 are arranged at positions different from each other in a back side portion of the seat cushion S1 as shown in FIG. 1, specifically arranged at six points. The arrangement positions of the breathing sensors 1 will be described in detail later.

Although the seating pressure is measured as the measurement values in the present embodiment, the present invention is not limited to this. That is, any physical amounts to be changed in accordance with breathing of the seated person on the seating surface Sf can serve as the measurement values. For example, a warp degree of the seating surface Sf or a temperature of the seating surface Sf may serve as the measurement values.

The ECU 2 is a device that determines the awakening state of the seated person on the basis of measurement results of the breathing sensors 1. As a method of determining the awakening state of the seated person on the basis of the measurement results of the breathing sensors 1 (that is, measurement results of the seating pressure), known determination methods can be utilized. As one example, in a waveform indicating a periodic change of the seating pressure, a period (in other words, an interval between peaks) may be obtained, and the awakening state of the seated person may be determined by whether or not a value of the interval is within a normal range.

A hardware configuration of the ECU 2 will be outlined. A controller 3 and a memory 4 are provided as main constituent elements of the ECU 2. The controller 3 is formed by a signal processor (not shown), an A/D converter, and an arithmetic instrument. The controller 3 receives signals indicating the measurement results from the breathing sensors 1, executes signal processing and A/D conversion processing to the signals, and then executes predetermined arithmetic processing. The controller 3 determines the awakening state of the seated person through arithmetic processing for state determination.

The controller 3 according to the present embodiment determines the awakening state on the basis of the measurement results of some sensors 1 among the plural breathing sensors 1. Thereby, in the present embodiment, a load of the arithmetic processing is reduced and more appropriate determination results can be obtained in comparison to a case where the awakening state is determined by using the measurement results of all the plural breathing sensors 1. Such an effect is an exceptional effect realized by the present system 10. Hereinafter, the configuration for realizing the effect will be described in detail.

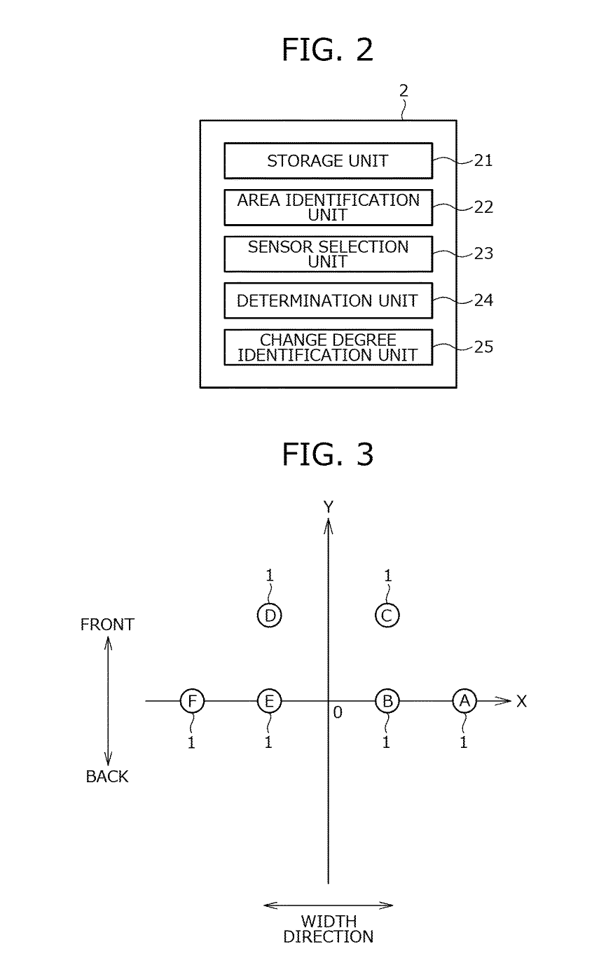

As the configuration for realizing the above effect, first of all, the configuration of the ECU 2 will be described once again from a functional aspect thereof with reference to FIG. 2. FIG. 2 is a block diagram showing the configuration of the ECU 2 from the functional aspect thereof. The ECU 2 has various functional units relating to state determination, specifically, a storage unit 21, an area identification unit 22, a sensor selection unit 23, a determination unit 24, and a change degree identification unit 25 shown in FIG. 2. The storage unit 21 is formed by the memory 4 of the ECU 2, and stores data required for selecting sensors actually used at the time of state determination among the plural breathing sensors 1 (hereinafter, referred to as the sensor selection data).

Upon describing the sensor selection data, the arrangement positions of the breathing sensors 1 will be described with reference to FIG. 3. FIG. 3 is a plan diagram showing arrangement of the breathing sensors 1. In the present embodiment, the plural breathing sensors 1 are arranged in such a manner that coordinates of the arrangement positions of the breathing sensors 1 are different from each other in a XY coordinate space. The XY coordinate space indicates a two dimensional coordinate space fixed when both the width direction and the front to back direction serve as the axial directions of the coordinate axes. Regarding coordinates of the origin position in the coordinate space, the X coordinate thereof is a center position of the seating surface Sf in the width direction and the Y coordinate thereof is a position on the slightly back side of the center position of the seating surface Sf in the front to back direction.

The plural breathing sensors 1 are arranged and divided into the front side and the back side as shown in FIG. 3. Specifically, the four breathing sensors 1 are arranged on the more back side and the two breathing sensors 1 are arranged on the more front side, so as to be placed respectively in a row along the width direction. The plural breathing sensors 1 are arranged bilaterally symmetrically with respect to the Y axis (in other words, the center position of the seating surface Sf in the width direction). That is, in the back side portion of the seat cushion S1, the four breathing sensors 1 are arranged symmetrically with respect to the Y axis in a more back side region, and the two breathing sensors 1 are arranged symmetrically with respect to the Y axis in a more front side region. The configuration that the arrangement number of the sensors in the back side region is more than the arrangement number of the sensors in the front side region in such a way reflects that the gravity center of the seated person is frequently positioned on the more back side on the seating surface Sf in general.

Hereinafter, the six breathing sensors 1 will be called the sensor A, the sensor B, the sensor C, the sensor D, the sensor E, and the sensor F for distinguishing the breathing sensors 1. The coordinates of the arrangement positions of the breathing sensors 1 are as follows.

(Arrangement position of sensor A)=(Xa, 0) wherein Xa is an arbitrary real number which is greater than 0.

(Arrangement position of sensor B)=(Xb, 0) wherein Xb is a real number which is greater than 0 and smaller than Xa.

(Arrangement position of sensor C)=(Xb, Yc) wherein Yc is an arbitrary real number which is greater than 0.

(Arrangement position of sensor D)=(−Xb, Yc)

(Arrangement position of sensor E)=(−Xb, 0)

(Arrangement position of sensor F)=(−Xa, 0)

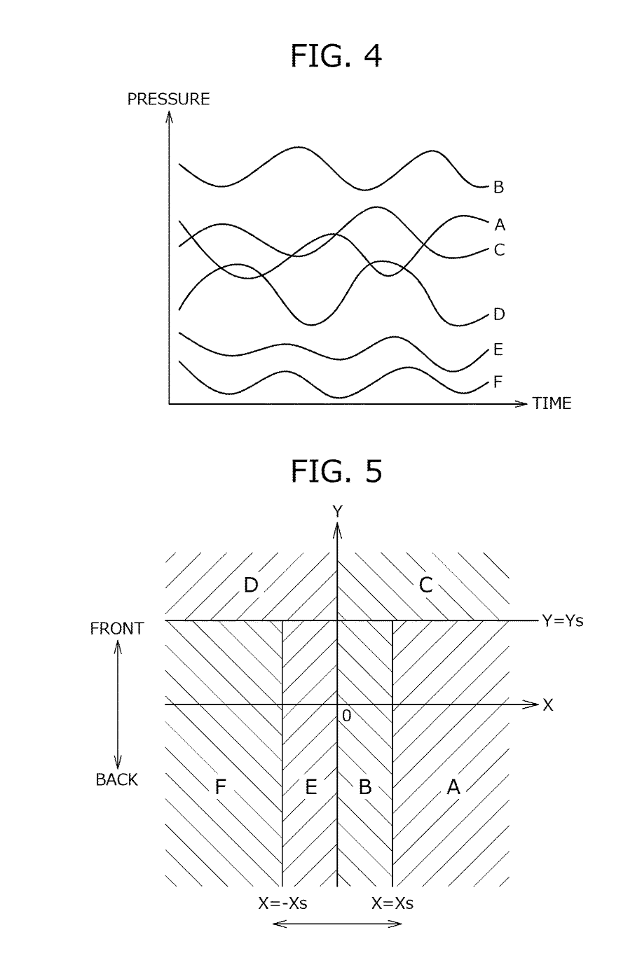

Since the arrangement positions of the breathing sensors 1 are different as described above, the seating pressure respectively measured by the six breathing sensors 1 is also different. Specifically speaking, when the breathing sensors 1 measure the seating pressure in a state where the seated person sits on the vehicle seat S in a certain seating state (strictly, a seated position or a seating posture), waveforms showing measurement results (hereinafter, referred to as the breathing waveforms) are different between the sensors as shown in FIG. 4. FIG. 4 is a diagram showing the breathing waveforms for the breathing sensors 1.

The breathing waveforms of the breathing sensors 1 are different between the sensors as described above, and varied in accordance with the seating state of the seated person. Meanwhile, as the breathing sensor 1 actually used at the time of state determination, there is a need for selecting a sensor whose breathing waveform having a waveform pattern which reflects a breathing action of the seated person more (hereinafter, referred to as the reflection sensor) among the six breathing sensors 1. Thus, the present embodiment makes clear which sensor corresponds to the reflection sensor among the six breathing sensors 1 when the seating state is changed, and the seating surface Sf is divided into areas on the basis of a result thereof.

More specifically speaking, in the present embodiment, the seating surface Sf is partitioned into plural areas in the XY coordinate space as shown in FIG. 5. FIG. 5 is a diagram showing positions of the areas when the seating surface Sf is partitioned into the plural areas. Each area is associated with one of the breathing sensors 1. Speaking in more detail, each area is associated with the breathing sensor 1 of the same reference sign as the reference sign given to each area in FIG. 5. For example, the area A is associated with the sensor A.

Relationships between the areas and the breathing sensors 1 associated with the areas will be described. When the coordinates of the gravity center position of the seated person in the XY coordinate space are placed in a certain area, the breathing sensor 1 associated with the certain area serves as the reflection sensor at the time point. That is, each area in the seating surface Sf shown in FIG. 5 shows which breathing sensor 1 serves as the reflection sensor when the coordinates of the gravity center position of the seated person is placed in the area (in other words, the breathing sensor 1 serving as the reflection sensor and an application range thereof). Data showing the association relationship between the range of each area and the breathing sensor 1 serving as the reflection sensor when the coordinates of the gravity center position are placed in the area is generated, and stored in the storage unit 21 as the sensor selection data. Hereinafter, the above association relationship will be described in detail.

The above association relationship is set by associating each of the areas when the seating surface is partitioned into the plural areas with some of the six breathing sensors 1. Specifically speaking, the association relationship is set by associating each of the areas with the breathing sensor 1 serving as the reflection sensor when the coordinates of the gravity center position of the seated person are placed in the area. Now, a method of identifying the reflection sensor will be described. A sensor having the highest “reflection degree of the breathing action on the breathing waveform” among the six breathing sensors 1 when the gravity center position of the seated person is placed in a certain area is identified as the reflection sensor. The “reflection degree of the breathing action on the breathing waveform” indicates a degree when the breathing action is reflected on the breathing waveform (measurement result of the seating pressure).

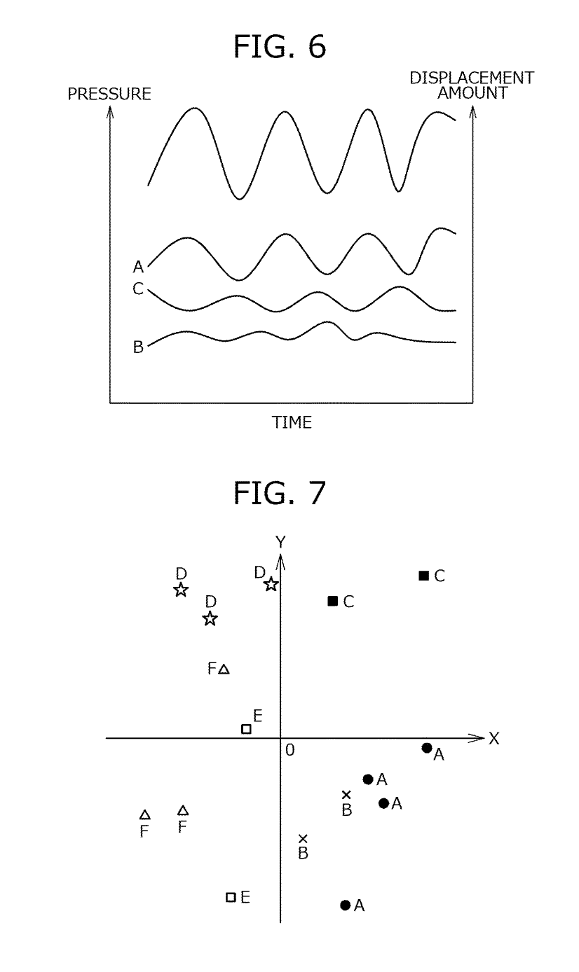

The method of identifying the reflection sensor will be described in more detail. In a state where the gravity center position of the seated person is placed in a certain area, the seating pressure is measured by the breathing sensors 1 and a physical amount to be remarkably changed in accordance with the breathing action of the seated person such as a displacement amount of a chest portion of the seated person (that is, an expanding amount of the chest portion) is measured by another sensor. After finishing measurement, breathing waveforms obtained from the measurement results of the breathing sensors 1 and a waveform obtained from a measurement result of the displacement amount of the chest portion (hereinafter, referred to as the chest portion displacement waveform) are compared. When both the waveforms are compared, as shown in FIG. 6, the breathing waveform whose waveform pattern is the closest to the chest portion displacement waveform is identified among the breathing waveforms for the breathing sensors 1. Such a breathing waveform corresponds to the breathing waveform having the highest reflection degree of the breathing action of the seated person, and the breathing sensor 1 obtaining this breathing waveform corresponds to the reflection sensor. FIG. 6 is a diagram showing the chest portion displacement waveform and the breathing waveforms for the breathing sensors 1. In the diagram, for easy understanding of the diagram, only the breathing waveforms of the sensors A to C among the six breathing sensors 1 are shown, and the breathing waveforms of the sensors D to F are omitted.

With the above method of identifying, by experimentally making clear the reflection sensor when the gravity center position of the seated person is changed (strictly speaking, the breathing sensor 1 corresponding to the reflection sensor), a result shown in FIG. 7 is obtained. FIG. 7 is a diagram experimentally making clear the reflection sensor when the coordinates of the gravity center position are changed, each plot in the diagram shows the gravity center position, and the reference sign displayed beside each plot shows the breathing sensor 1 corresponding to the reflection sensor.

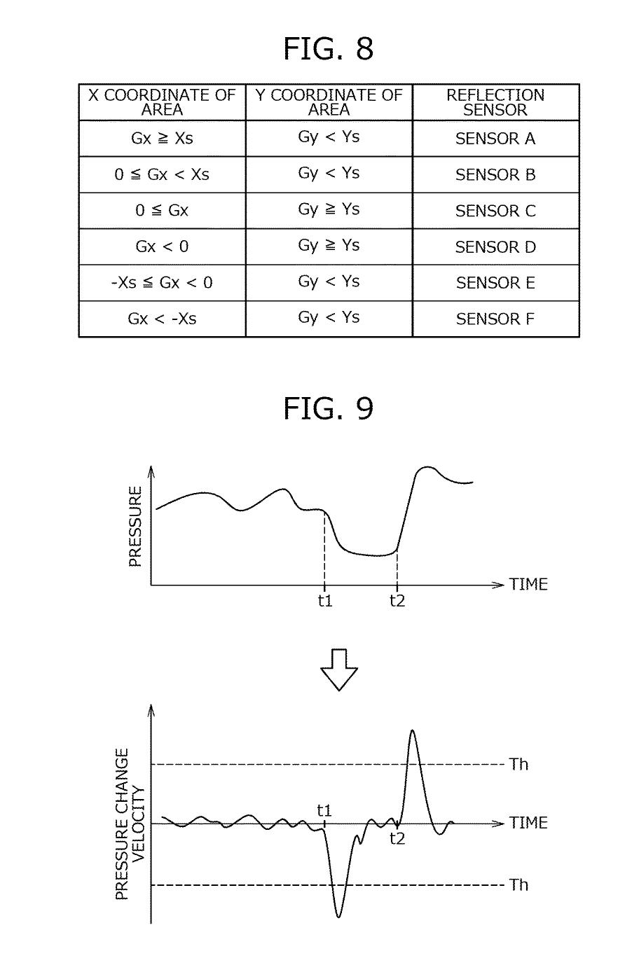

By dividing into areas on the basis of the experiment result shown in FIG. 7, the seating surface Sf is partitioned into the plural areas as shown in FIG. 5 (six areas in FIG. 5). Then, data showing the association relationship between each area and the breathing sensor serving as the reflection sensor when the coordinates of the gravity center position are placed in the area, that is, the selection data is generated and stored in the storage unit 21. In the present embodiment, data showing the association relationships shown in FIG. 8 is stored as the selection data. FIG. 8 is a diagram showing the association relationships between the areas and the reflection sensor, and specifically, shows coordinates (the X coordinate and the Y coordinate) showing the range of each area and the breathing sensor 1 corresponding to the reflection sensor when the coordinates of the gravity center position are placed in the area. Values Xs and Ys in the diagram indicate coordinate values for setting boundary lines of the areas, the coordinate values being set from for example the experiment result shown in FIG. 7.

The division of the seating surface Sf into the areas according to the present embodiment will be described in more detail with reference to FIG. 8. The seating surface Sf is partitioned in such a manner that two or more areas exist respectively in both the width direction and the front to back direction. In other words, the selection data stored by the storage unit 21 shows the association relationship set by associating each area when the seating surface Sf is partitioned in such a manner that two or more areas exist respectively in the width direction and the front to back direction with the breathing sensor 1 serving as the reflection sensor when the coordinates of the gravity center position are placed in the area.

In the present embodiment, the seating surface Sf is partitioned in such a manner that the number of the areas positioned on the back side in the front to back direction is more than the number of the areas positioned on the front side. In other words, the selection data stored by the storage unit 21 shows the association relationship set by associating each area when partitioning in such a manner that the number of the areas positioned on the back side is more than the number of the areas positioned on the front side with the breathing sensor 1 serving as the reflection sensor when the coordinates of the gravity center position are placed in the area. The configuration that the number of the areas positioned on the back side is more than the number of the areas positioned on the front side as described above reflects that the gravity center of the seated person is frequently positioned on the back side of the seating surface Sf in general.

Further, in the present embodiment, the seating surface Sf is partitioned in such a manner that the areas exist symmetrically with respect to the center position of the seating surface Sf in the width direction. In other words, the selection data stored by the storage unit 21 shows the association relationship set by associating each area when the seating surface Sf is partitioned in such a manner that the areas exist symmetrically with respect to the center position of the seating surface Sf in the width direction with the breathing sensor 1 serving as the reflection sensor when the coordinates of the gravity center position are placed in the area.

In the present embodiment, as shown in FIG. 5, the four areas are arranged symmetrically with respect to the Y axis in the region on the back side (specifically, a back end portion) of the seating surface Sf, and the two areas are arranged symmetrically with respect to the Y axis in the region on the more front side (region positioned on the front side of the back end portion).

Furthermore, in the present embodiment, the seating surface Sf is partitioned in such a manner that the areas closer to the center position of the seating surface Sf (such as the area B and the area E in FIG. 5) among the four areas placed side by side in the width direction on the more back side are narrower than the areas more distant from the center position (such as the area A and the area F in FIG. 5). In other words, the selection data stored by the storage unit 21 shows the association relationship set by associating each area when the seating surface Sf is partitioned in such a manner that width of the area in the vicinity of the center position of the seating surface Sf (length in the width direction) is shorter than width of the area positioned on the outer side with the breathing sensor 1 serving as the reflection sensor when the coordinates of the gravity center position are placed in the area.

Next, the area identification unit 22, the sensor selection unit 23, the determination unit 24, and the change degree identification unit 25 will be described. These function units are formed by the controller 3, more specifically, the signal processor, the A/D converter, and the arithmetic instrument mounted on the controller 3, and an arithmetic program executed by the arithmetic instrument.

The area identification unit 22 calculates the coordinates of the gravity center position of the seated person in the XY coordinate space. Specifically speaking, in the present embodiment, the area identification unit 22 calculates the coordinates of the gravity center position on the basis of the measurement results of the sensors arranged at the positions sandwiching the Y axis (strictly speaking, the sensors B to E) among the six breathing sensors 1. Speaking in more detail, when the measurement result of the sensor B is Pb, the measurement result of the sensor C is Pc, the measurement result of the sensor D is Pd, and the measurement result of the sensor E is Pe, the X coordinate Gx and the Y coordinate Gy of the gravity center position are calculated by the following equations.

Gx={m×(Pb+Pc)+(−m)×(Pd+Pe)}/(Pb+Pc+Pd+Pe)

Gy={n×(Pc+Pd)}/(Pb+Pc+Pd+Pe)

m=Xb, n=Ye

After calculating the coordinates of the gravity center position, the area identification unit 22 identifies in which area the coordinates of the gravity center position are placed among the six areas made by partitioning as described above.

The sensor selection unit 23 selects the sensor associated with the area identified by the area identification unit 22 among the six breathing sensors 1, that is, the sensor associated with the area to which the gravity center position belongs on the basis of the association relationship shown by the selection data which is stored by the storage unit 21. The determination unit 24 determines the awakening state of the seated person on the basis of the measurement result of only the sensor selected by the sensor selection unit 23.

The change degree identification unit 25 identifies a change degree of the measurement result of the breathing sensor 1 selected by the sensor selection unit 23. The change degree indicates a change amount per unit time of the seating pressure measured by the breathing sensor 1 which is selected by the sensor selection unit 23. Specifically, the change degree indicates a velocity component obtained by first-order differentiating the breathing waveform showing a temporal change of the seating pressure (hereinafter, referred to as the pressure change velocity). The change degree is radically changed as shown in FIG. 9 for example when the seated person sitting on the vehicle seat S moves his/her body or the like. FIG. 9 is a diagram showing the measurement result of the breathing sensor 1 and the change degree thereof. An upper portion of the diagram shows the waveform showing the measurement result (breathing waveform), and a lower portion of the diagram shows a waveform of the change degree (pressure change velocity).

The change degree identification unit 25 determines whether or not a magnitude of the identified change degree (pressure change velocity) exceeds a threshold value. Speaking in detail with reference to FIG. 9, the change degree identification unit 25 identifies the pressure change velocity serving as the change degree of the measurement result of the breathing sensor 1 selected by the sensor selection unit 23 at fixed intervals. When the seated person moves his/her body at times t1 and t2, the measurement result of the breathing sensor 1 is radically changed, and the pressure change velocity is accordingly radically increased (or radically decreased).

Meanwhile, the change degree identification unit 25 determines whether or not the magnitude of the pressure change velocity exceeds the threshold value (Th in FIG. 9) each time the pressure change velocity is identified. In a case shown in FIG. 9, the magnitude of the pressure change velocity exceeds the threshold value Th in accordance with body movement of the seated person at the times t1 and t2. Thus, the change degree identification unit 25 determines that the magnitude of the pressure change velocity exceeds the threshold value Th.

In the present embodiment, as the change degree of the measurement result of the breathing sensor 1, the velocity component (pressure change velocity) obtained by first-order differentiating the breathing waveform which is obtained from the measurement result is used. However, the present invention is not limited to this. For example, an acceleration component (pressure change acceleration) obtained by further differentiating the waveform of the pressure change velocity may be used.

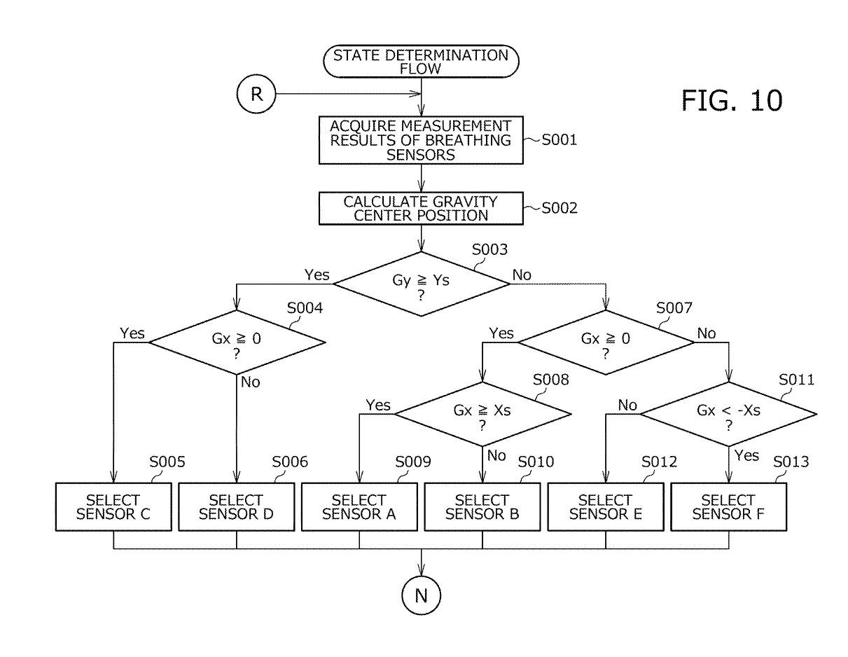

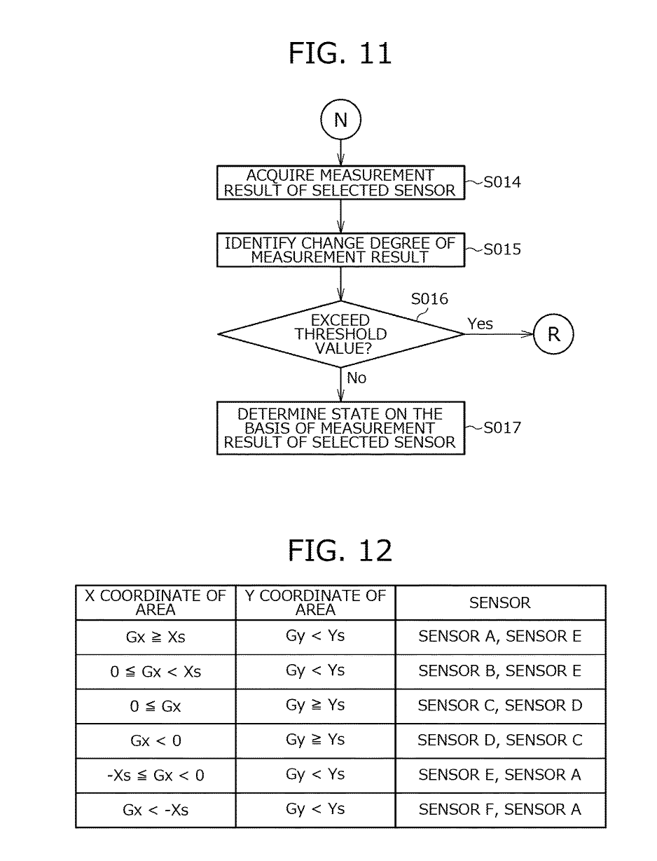

Next, a state determination flow implemented by the present system 10 described above will be described with reference to FIGS. 10 and 11. FIGS. 10 and 11 show a flow of the state determination flow.

Before the state determination flow, the seating surface Sf of the vehicle seat S is partitioned into six areas as described above. Before the state determination flow, each of the above six areas is associated with the breathing sensor 1 serving as the reflection sensor when the coordinates of the gravity center position are placed in the area. Further, before the state determination flow, the data showing the association relationship between each area and the breathing sensor 1, that is, the selection data is generated and stored in the memory 4 of the ECU 2.

Meanwhile, the state determination flow is started at a time point when the seated person sits on the vehicle seat S. When the state determination flow is started, first of all, each of the six breathing sensors 1 starts measurement, and the controller 3 acquires the measurement results from the breathing sensors 1 (S001). At this time, the controller 3 acquires only the measurement results of the four breathing sensors 1 (specifically, the sensors B to E) among the six breathing sensors 1. The controller 3 acquires the measurement results for a predetermined time period (such as for 30 seconds) regarding the respective four breathing sensors 1, and then calculates an average value of the measurement results for the predetermined time period.

Next, the controller 3 calculates the coordinates of the gravity center position of the seated person by using the measurement results of the four breathing sensors 1 acquired in the former step S001 (strictly speaking, the average value for the predetermined time period) (S002). On the basis of the calculation result, the controller 3 identifies in which area the coordinates of the gravity center position are placed (S003, S004, S007, S008, S011). After that, the controller 3 reads out the selection data stored in the storage unit 21, and selects the breathing sensor 1 associated with the area to which the gravity center position belongs from the association relationship shown by the data (S005, S006, S009, S010, S012, S013).

After that, the controller 3 determines the awakening state of the seated person on the basis of the measurement result of one sensor selected among the six breathing sensors 1. However, in the present embodiment, in a case where the seated person moves his/her body or the like and the gravity center position is changed after selection of the sensor, selection of the sensor is implemented again, so that the breathing sensor 1 used at the time of state determination is re-selected. Such a flow will be described with FIG. 11. After selection of the sensor, the measurement result of the selected breathing sensor 1 is acquired at fixed intervals (S014). Meanwhile, the controller 3 (specifically, the change degree identification unit 25) identifies the change degree of the measurement result of the above breathing sensor 1, that is, the pressure change velocity (S015). In a case where the magnitude of the identified pressure change velocity does not exceed the threshold value Th (No in S016), the controller 3 determines the awakening state of the seated person on the basis of the measurement result of the breathing sensor 1 selected before identifying the pressure change velocity (S017).

Meanwhile, in a case where the magnitude of the identified pressure change velocity exceeds the threshold value Th (Yes in S016), the flow returns to Step S001 and the series of above steps will be started over from the beginning. That is, the controller 3 re-identifies in which area the coordinates of the gravity center position of the seated person are placed, and accordingly re-selects the breathing sensor 1 associated with the identified area. After that, the controller 3 determines the awakening state of the seated person on the basis of the measurement result of the re-selected breathing sensor 1 (S017).

As described above, in the state determination flow according to the present embodiment, one sensor is selected among the six breathing sensors 1 for the purpose of reducing the load of the arithmetic processing relating to state determination. At the time of selection of the sensor, a proper breathing sensor 1 for determining the awakening state in consideration with the seated position to be used of the seated person, strictly speaking, the breathing sensor 1 corresponding to the reflection sensor is selected. With such a state determination flow, when only one breathing sensor 1 is selected among the six breathing sensors 1 as a sensor actually used at the time of determination of the awakening state, the sensor can be easily and properly selected.

In the present embodiment, in a case where the seated person moves and the gravity center position is changed after selection of the sensor, the sensor is re-selected, so that the sensor associated with the area to which the gravity center position after the change belongs is selected again. Thereby, even after the gravity center position is changed, the awakening state of the seated person can be properly determined. Meanwhile, in a case where the gravity center position is not changed, the measurement result of the breathing sensor 1 which has been used until then is continuously utilized, and the awakening state is determined on the basis of the measurement result. Thereby, in a case where the gravity center position is not changed, there is no need for re-selecting the sensor, and time and labor of re-selecting can be omitted.

<<State Determination System According to Modified Example>>

In the embodiment described above, the awakening state of the seated person is determined on the basis of the measurement result of one breathing sensor 1 among the six breathing sensors 1. However, the present invention is not limited to this. That is, a configuration example that the awakening state is determined on the basis of the measurement results of two breathing sensors 1 among the six breathing sensors 1 (hereinafter, referred to as the modified example) can be considered. Hereinafter, a state determination system according to the modified example will be described. Hereinafter, only contents which are different from the state determination system according to the already-described embodiment (that is, the present system 10) will be described, and description of contents which are common to the present system 10 will be omitted.

In the modified example, each area when the seating surface Sf is partitioned into six areas is associated with a breathing sensor 1 serving as the reflection sensor when the coordinates of the gravity center position are placed in the area, and also associated with a breathing sensor 1 paired with the reflection sensor together. That is, in the modified example, each area is associated with two breathing sensors 1. In other words, selection data stored in the storage unit 21 in the modified example shows association relationships set by associating each area with the two breathing sensors 1.

The breathing sensor 1 paired with the reflection sensor is a sensor among the five breathing sensors 1 excluding the reflection sensor, the sensor having the highest relativity between a measurement result of the sensor and a measurement result of the reflection sensor. Specifically, the sensor is the breathing sensor 1 paired with the breathing sensor 1 corresponding to the reflection sensor among the two breathing sensors 1 associated with each area shown in FIG. 12. FIG. 12 is a diagram showing the association relationships between each area and the breathing sensors 1.

The above association relationships according to the modified example will be described in more detail with reference to FIGS. 5 and 12. In FIG. 5, when the coordinates of the gravity center position of the seated person are placed in the area A, the sensor A corresponds to the reflection sensor, and the sensor E corresponds to the paired sensor. Similarly, when the gravity center position is placed in the area B, the sensor B corresponds to the reflection sensor, and the sensor E corresponds to the paired sensor. When the gravity center position is in the area C, the sensor C corresponds to the reflection sensor, and the sensor D corresponds to the paired sensor. When the gravity center position is placed in the area D, the sensor D corresponds to the reflection sensor, and the sensor C corresponds to the paired sensor. When the gravity center position is placed in the area E, the sensor E corresponds to the reflection sensor, and the sensor A corresponds to the paired sensor. When the gravity center position is placed in the area F, the sensor F corresponds to the reflection sensor, and the sensor A corresponds to the paired sensor.

A positional relationship between the reflection sensor and the sensor paired with the reflection sensor with respect to coordinates of the gravity center position at a certain time will be described. The coordinates of the gravity center position at the certain time exist between both the sensors in the width direction. That is, the selection data stored in the storage unit 21 in the modified example shows an association relationship set by associating each area with two breathing sensors 1 whose coordinates of sensor arrangement positions straddle the coordinates of the gravity center position in the width direction when the coordinates of the gravity center position are placed in the area.

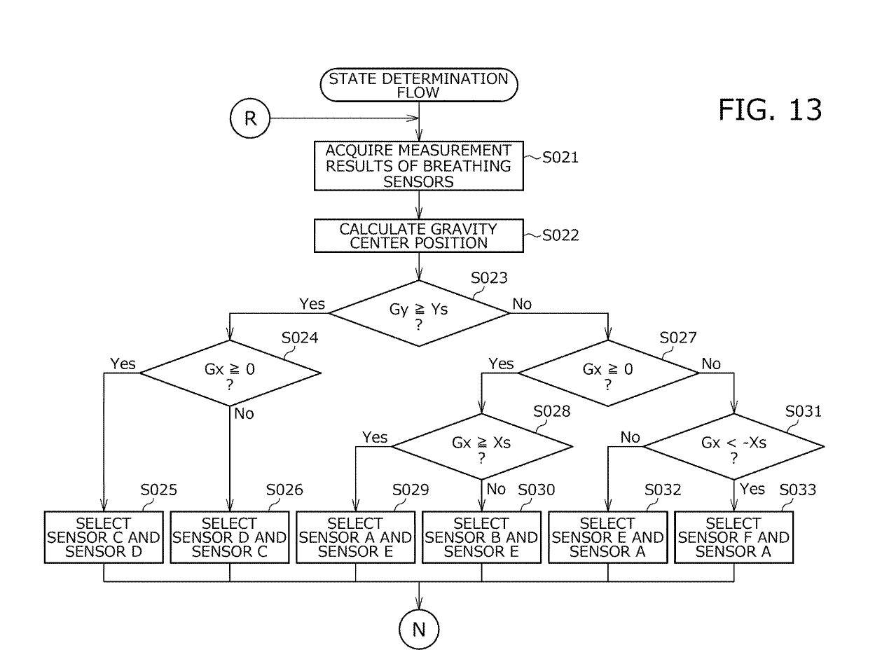

Next, a state determination flow according to the modified example will be described with reference to FIG. 13. FIG. 13 is a diagram showing a flow of the state determination flow according to the modified example, the diagram being associated with FIG. 10. Almost all the steps in the state determination flow according to the modified example are similar to the state determination flow according to the already-described embodiment as shown in FIG. 13. That is, when the state determination flow is started, each of the six breathing sensors 1 starts measurement, and the controller 3 acquires the measurement results from the four breathing sensors 1 (strictly speaking, the sensors B to E) (S021). The controller 3 calculates the coordinates of the gravity center position of the seated person on the basis of the measurement results of the breathing sensors 1 acquired in the former step S021 (S022). On the basis of the calculation result, the controller 3 identifies in which area the coordinates of the gravity center position are placed (S023, S024, S027, S028, S031). After that, the controller 3 reads out the selection data stored in the storage unit 21, and selects two breathing sensors 1 associated with the area to which the gravity center position belongs (S025, S026, S029, S030, S032, S033).

In the modified example, on the basis of the measurement results of the two breathing sensors 1 selected among the six breathing sensors 1, the controller 3 determines the awakening state of the seated person. Strictly speaking, the controller 3 according to the modified example averages the measurement results of the selected two breathing sensors 1, and determines the awakening state on the basis of the average value.

As described above, in the state determination flow according to the modified example, two sensors are selected among the six breathing sensors 1, and the awakening state of the seated person is determined on the basis of the two sensors. With such a state determination flow, even when an abnormality is caused in one of the selected two breathing sensors 1 but the other sensor is normal, the awakening state of the seated person can be properly determined on the basis of the measurement result of the sensor.

### Other Embodiment

In the embodiment described above, one example of the configuration and the actions of the state determination system of the present invention is described. However, the above embodiment is only one example for facilitating understanding of the present invention but never limit the present invention. That is, the present invention can be modified or improved without departing from the gist thereof, and equivalents thereof are included in the present invention as a matter of course.

Although the vehicle seat S is taken as one example of the seat in the above embodiment, the present invention is not limited to this. The present invention can also be applied to a case of determining a physiological state of a seated person sitting on a general seat (seating) to be utilized in for example a company, a school, a factory, an entertainment facility such as a theater, a hospital, a residence, and the like. In addition, the present invention can also be applied to a case where a physiological state of a seated person sitting on a seat mounted on a means of transportation other than the vehicle.

In the above embodiment, the four breathing sensors 1 are arranged in the more back side region in the back side portion of the seat cushion S1 and the two breathing sensors 1 are arranged in the more front side region, respectively symmetrically with respect to the center position in the width direction of the seating surface Sf. However, the number and the arrangement positions of the breathing sensors 1 are not limited to the above contents. As long as the arrangement number of the breathing sensors 1 is more in the back side region and the breathing sensors 1 are arranged symmetrically with respect to the center position in the width direction of the seating surface Sf as in the above embodiment, a proper breathing sensor 1 can be selected as the sensor associated with the area to which the gravity center position belongs.

In the above embodiment, the seating surface Sf is divided into the areas as shown in FIG. 5. Specifically, the seating surface is divided into the areas in such a manner that two or more areas exist in each of the width direction and the front to back direction. However, the present invention is not limited to this. The seating surface may be divided into the areas in such a manner that two or more areas exist in at least one of the width direction and the front to back direction. When the seating surface is divided into the areas as in the above embodiment, the seating surface Sf is more finely partitioned, and the area to which the gravity center position belongs can be finely identified. As a result, when the breathing sensor 1 associated with the area to which the gravity center position belongs is selected, a more proper sensor can be selected.

In the above embodiment, the seating surface is divided into the areas in such a manner that the number of the areas positioned on the back side in the front to back direction is more than the number of the areas positioned on the front side. However, the present invention is not limited to this. The number of the areas positioned on the front side may be more than the number of the areas positioned on the back side, or the numbers may be the same. When the seating surface is divided into the areas as in the above embodiment, the number of the areas in the back side region of the seating surface Sf where the gravity center of the seated person is frequently positioned is more. Thus, the area to which the gravity center position belongs can be more finely identified. As a result, the sensor associated with the area to which the gravity center position belongs is selected, a more proper sensor can be selected.

In the above embodiment, the seating surface is divided into the areas in such a manner that the areas exist symmetrically with respect to the center position in the width direction of the seating surface Sf. However, the present invention is not limited to this. The arrangement positions of the areas may be in a non-symmetrical positional relationship in the width direction. When the seating surface is divided into the areas as in the above embodiment, symmetry of area arrangement is utilized, so that identification of the area to which the gravity center position belongs and selection of the sensor can be more readily performed.

In the above embodiment, the seating surface is divided into the areas in such a manner that the width of the area in the vicinity of the center position in the width direction of the seating surface Sf among the plural areas (specifically, the four areas) placed in a row in the width direction is shorter than the width of the area distant from the center position in the width direction. However, the present invention is not limited to this. The width of the area in the vicinity of the center position in the width direction may be longer than the width of the area distant from the center position in the width direction. When the seating surface is divided into the areas as in the above embodiment, the area closer to the center position in the width direction of the seating surface Sf among the areas placed side by side in the width direction is narrower. Thus, the area to which the gravity center position belongs can be more finely identified in the vicinity of the center position in the width direction. As a result, when the sensor associated with the area to which the gravity center position belongs is selected, a more proper sensor can be selected.

### REFERENCE SIGNS LIST

1: Breathing sensor (sensor)
2: ECU (determination device)
3: Controller
4: Memory
10: Present system (state determination system)
21: Storage unit
22: Area identification unit
23: Sensor selection unit
24: Determination unit
25: Change degree identification unit
S: Vehicle seat (seat)
S1: Seat cushion
Sf: Seating surface

1. A state determination system comprising:
sensors that measure measurement values to be changed in accordance with a physiological activity of a seated person on a seating surface of a seat; and
a determination device that determines a physiological state of the seated person on the basis of measurement results of the sensors, wherein
the plural sensors are arranged in such a manner that coordinates of arrangement positions of the sensors fixed when both width direction and front to back direction of the seat serve as axial directions of coordinate axes are different from each other, and
the determination device includes:
a storage unit that stores association relationships set by associating each of areas when the seating surface is partitioned into the plural areas with some of the plural sensors;
an area identification unit that identifies in which area coordinates of a gravity center position of the seated person fixed when both the directions serve as the axial directions of the coordinate axes are placed among the plural areas;
a sensor selection unit that selects some of the sensors associated with the area identified by the area identification unit among the plural sensors from the association relationships stored by the storage unit; and
a determination unit that determines the physiological state on the basis of the measurement results of the sensors selected by the sensor selection unit.

2. The state determination system according to claim 1, wherein
the storage unit stores the association relationships set by associating each of the areas when the seating surface is partitioned in such a manner that two or more areas exist in each of both the directions with some of the plural sensors.

3. The state determination system according to claim 2, wherein
the storage unit stores the association relationships set by associating each of the areas when the seating surface is partitioned in such a manner that the number of the areas positioned on back side in the front to back direction is more than the number of the areas positioned on front side with some of the plural sensors.

4. The state determination system according to claim 1, wherein
at least three or more of the sensors are arranged, and
the storage unit stores the association relationships set by associating each of the areas with two of the sensors.

5. The state determination system according to claim 4, wherein
the storage unit stores the association relationships set by associating each of the areas with two sensors among the plural sensors, the two sensors whose coordinates of the arrangement positions straddle the coordinates of the gravity center position in the width direction when the coordinates of the gravity center position are placed in each of the areas.

6. The state determination system according to claim 1, wherein
the storage unit stores the association relationships set by associating each of the areas when the seating surface is partitioned in such a manner that the areas exist symmetrically with respect to a center position of the seating surface in the width direction with some of the plural sensors.

7. The state determination system according to claim 1, wherein
the storage unit stores the association relationships set by associating each of the areas when the seating surface is partitioned in such a manner that length in the width direction of the area closer to a center position of the seating surface among at least three or more of the areas placed side by side in the width direction is shorter than the length of the area more distant from the center position with some of the plural sensors.

8. The state determination system according to claim 1, wherein
the number of the sensors arranged on back side in the front to back direction is more than the number of the sensors arranged on front side.

9. The state determination system according to claim 8, wherein
four of the sensors are arranged symmetrically with respect to a center position of the seating surface in the width direction on the back side in the front to back direction, and two of the sensors are arranged symmetrically with respect to the center position in the width direction on the front side.

10. The state determination system according to claim 1, wherein
the storage unit stores the association relationships set by associating each of the areas with a sensor among the plural sensors, the sensor of a higher reflection degree on the measurement result of the measurement value of the physiological activity when the coordinates of the gravity center position are placed in each of the areas.

11. The state determination system according to claim 1, wherein
the determination device further has a change degree identification unit that identifies a change degree of the measurement results of the sensors selected by the sensor selection unit,
when a magnitude of the change degree identified by the change degree identification unit exceeds a threshold value, the area identification unit re-identifies the area, and accordingly the sensor selection unit re-selects some of the sensors, and then the determination unit determines the physiological state on the basis of measurement results of only the re-selected sensors, and
when the magnitude of the change degree identified by the change degree identification unit falls below the threshold value, the determination unit determines the physiological state on the basis of the measurement results of only the sensors selected by the sensor selection unit before the change degree identification unit identifies the change degree.
